(19) United States
(12) Patent Application Publication
(10) Pub. No.: US 2008/0131406 A1
(43) Pub. Date: Jun. 5, 2008

(54) Angiotensin I Derivatives

(75) Inventor: Meng K. Sim, Singapore (SG)

Correspondence Address: MARSHALL, GERSTEIN & BORUN LLP, 233 S. WACKER DRIVE, SUITE 6300, SEARS TOWER, CHICAGO, IL, 60606, US

(73) Assignee: NATIONAL UNVERSITY OF SINGAPORE, Singapore (SG)

(21) Appl. No.: 11/814,235

(22) PCT Filed: Jan. 17, 2006
(86) PCT No.: PCT/SG06/00006

§ 371 Date: Jan. 4, 2008

Related U.S. Application Data
(60) Provisional application No. 60/645,167, filed on Jan. 18, 2005.

(51) Int. Cl. A61K 35/12; A61K 38/16; A61P 9/00

(52) U.S. Cl. 424/93.7; 514/12

(57) ABSTRACT

The present invention relates to angiotensin I derivatives. In particular, the present invention relates to the use of angiotensin I derivatives, excluding des-aspartate-angiotensin I, for the treatment and/or prevention of cardiac hypertrophy, and/or neointima formation.

### FIELD OF THE INVENTION

The present invention relates to angiotensin I derivatives. In particular, the present invention relates to angiotensin I derivatives but excluding des-aspartate-angiotensin I.

### BACKGROUND OF THE INVENTION

In the body, the peptide angiotensin I is converted to angiotensin III by aminopeptidases(s) and angiotensin converting enzyme, respectively, via the intermediate molecule des-aspartate-angiotensin I.

Des-aspartate-angiotensin I has been described for use in treatment and/or prevention of cardiac hypertrophy (U.S. Pat. No. 5,773,415), and neointima formation or restenosis (U.S. Pat. No. 6,100,237).

Angiotensin II is involved in cardiac hypertrophy and neointima formation. Exogenously-administered angiotensin II potentiates cardiac hypertrophy (Dostal and Baker, Am. J. Hypertens., 5:276-280 (1991)), and neointima formation (Osterrieder et al, Hypertension, 18:II-60-II-64 (1991); Daemen et al, Circ. Res., 68:450-456 (1991)).

The actions of angiotensin IV, a secondary metabolite of angiotensin II, are less well known. Angiotensin IV has recently been shown to act on a subtype of angiotensin receptor, which is different from the known AT1 and AT2 receptors (Swanson et al, Regul. Pept., 40:409-419 (1992)). This receptor is named AT4 receptor and has been shown to regulate cognitive function in the brain and possibly neuronal development (von Bohlen and Halbach, Cell Tissue Res., 311:1-9 (2003)). Its role, if any, in cardiac hypertrophy, is indeterminate.

Two studies reported contradictory effects. A study by Baker and Aceto (Am. J. Physiol., 259:H610-H618 (1990)) showed that angiotensin IV inhibited the stimulatory effects of angiotensin II on protein synthesis and cell growth in cultures of embryonic chick myocytes. A later study by Wang et al (Clin. Sci., 88:557-562 (1995)) showed that both angiotensin II and angiotensin IV stimulated the DNA and RNA synthesis in quiescent rabbit cardiac fibroblast, and combination of the two peptides resulted in additive stimulation of RNA synthesis.

These in vitro studies provide no indication as to the action of angiotensin IV (AT4) on cardiac hypertrophy in an intact mammalian species. Similarly, there is a paucity of information on the effect of angiotensin IV on neointima formation. A study by Moeller et al (Regul. Pept., 83:25-30 (1999)) reported an upregulation of AT4 receptors in the neointima and media of endothelial denuded rabbit carotid artery. However, as in cardiac hypertrophy, the exact role of angiotensin IV in neointima formation remains unknown.

### SUMMARY OF THE INVENTION

The present invention addresses the problems above and provides new uses and/or composition(s) of derivative(s) of angiotensin I. In particular, the present invention provides new uses of derivative(s) of angiotensin I, with the exclusion of des-aspartate-angiotensin I. More in particular, the derivatives of angiotensin I, with the exclusion of des-aspartate-angiotensin I, are used for the treatment and/or prevention of cardiac hypertrophy, and/or neointima formation, including restenosis, in a subject or human patient in need of such treatment or prevention.

Accordingly, there is provided a method for the treatment and/or prevention of cardiac hypertrophy and/or neointima formation in a subject in need of such treatment and/or prevention comprising administering to the patient an effective amount of at least one derivative of angiotensin I, with the exclusion of des-aspartate-angiotensin I.

There is also provided a pharmaceutical composition comprising an effective amount of at least one derivative of angiotensin I, with the exclusion of des-aspartate-angiotensin I, and at least one pharmaceutically acceptable carrier, excipient, diluent and/or adjuvant. The composition is preferably for use in the treatment and/or prevention of cardiac hypertrophy and/or neointima formation in a subject in need of such treatment and/or prevention comprising administering to the patient. The patient may be human. In particular, the neointima formation may comprise restenosis. There is also provided at least one derivative of angiotensin I, with the exclusion of des-aspartate-angiotensin I, for use in medicine. The derivative according to the invention is preferably for use in the treatment and/or prevention of cardiac hypertrophy and/or neointima formation in a subject in need of such treatment. The patient may be human. In particular, the neointima formation may comprise restenosis.

There is also provided the use of at least one derivative of angiotensin I, with the exclusion of des-aspartate-angiotensin I, for the preparation of a medicament for the treatment and/or prevention of cardiac hypertrophy, and/or neointima formation in a subject in need of such treatment or prevention. The patient may be human. In particular, the neointima formation may comprise restenosis.

Further, there is provided a kit comprising at least one derivative of angiotensin I with the exclusions of des-aspartate-angiotensin I, wherein the kit is for the treatment and/or prevention of cardiac hypertrophy and/or neointima formation. The kit may further comprise information, illustration and/or indication pertaining to the use.

The derivative of angiotensin I may be a derivative, homologue, analogue and/or chemical equivalent of angiotensin I. For example, the derivative may be a derivative, homologue, analogue and/or chemical equivalent of angiotensin IV. In particular, the at least one derivative is angiotensin IV.

The derivative may be prepared, used and/or administered in an effective amount. The effective amount may be 10 to 500 μg/kg/day or 50 to 250 μg/kg/day. In particular, the derivative is prepared, used and/or administered in an effective amount of about 150 μg/kg/day for the treatment and/or prevention of cardiac hypertrophy, and in about 200 μg/kg/day for the treatment and/or prevention of neointima formation and/or restenosis.

The derivative, medicament or the pharmaceutical composition according to the invention may be administered in solid or liquid form.

The derivative may be administered together with a pharmaceutically acceptable carrier, excipient, diluent and/or adjuvant. Further, the derivative may be administered in conjunction with at least one pharmaceutical agent. The at least one pharmaceutical agent is an angiotensin converting enzyme inhibitor, an angiotensin receptor antagonist, and/or at least one type of stem cell.

### DETAILED DESCRIPTION

Bibliographic references mentioned in the present specification are for convenience listed in the form of a list of references and added at the end of the examples. The whole content of such bibliographic references is herein incorporated by reference.

The present invention relates to a new use in medicine for at least one derivative of angiotensin I. In particular, the invention relates to the use in medicine of at least one derivative of angiotensin I, with the exclusion of des-aspartate-angiotensin I. The derivative used in the present invention may be a derivative, homologue, analogue and/or chemical equivalent of angiotensin I. For example, a derivative, homologue, analogue and/or chemical equivalent of angiotensin IV. In particular, the derivative may be angiotensin IV. More in particular, there is provided the use of at least one derivative of angiotensin I, with the exclusion of des-aspartate-angiotensin I, for the treatment and/or prevention of cardiac hypertrophy and/or neointima formation in a subject in need of such treatment and/or prevention.

The effects of an example of angiotensin IV, which is a derivative of angiotensin I, on cardiac hypertrophy, and/or neointima formation/restenosis in a rat as an example of a mammalian subject following experimentally-induced cardiac hypertrophy and/or neointima formation or restenosis were determined. The inventors surprisingly found that at least one derivative of angiotensin I, other than des-aspartate-angiotensin I, prevented or attenuated or decreased the cardiac hypertrophy, and/or neointima formation/restenosis.

Animal models for studying cardiac hypertrophy, and/or neointima formation or restenosis, including small mammals such as the rat are well accepted in the art (Everette et al, Hypertension, 23:587-593 (1994); Indolfi et al, Circulation, 92:1230-1235 (1995)). The inventors have obtained surprising results that a derivative of angiotensin I such as angiotensin IV, was capable of preventing or ameliorating cardiac hypertrophy, and/or neointima formation/restenosis.

Accordingly, one aspect of the present invention relates to the use of derivatives of angiotensin I, with the exception of des-aspartate-angiotensin I, for the treatment and/or prevention of cardiac hypertrophy, and/or neointima formation or restenosis. Preferably, the at least one derivative of angiotensin I is administered in the form of an affective amount for the treatment and/or prevention of cardiac hypertrophy, and/or neointima formation or restenosis.

Another aspect of the present invention is the use of an effective amount of a derivative of angiotensin I, with the exception of des-aspartate-angiotensin I, for the preparation of a medicament for the treatment and/or prevention of cardiac hypertrophy, and/or neointima formation or restenosis. The medicament may be administered in conjunction with at least one pharmaceutically acceptable carrier, excipient, diluent and/or adjuvant. The medicament may also be administered in conjunction with a further pharmaceutical agent (or compound).

While derivatives of angiotensin I, with the exception of des-aspartate-angiotensin I, have been studied for in vitro binding or receptors, there is no indication or suggestion in the state of the art for the use of derivatives of angiotensin I according to the invention for use in medicine. In particular, there is no indication or suggestion in the art for the use of at least one derivative of angiotensin I, with the exception of des-aspartate-angiotensin I, for use in the treatment and/or prevention of cardiac hypertrophy and/or neointima formation in a subject in need of such treatment.

Another aspect of the invention is a kit comprising a derivative of angiotensin I other than des-aspartate-angiotensin I. In particular, the kit is for the treatment or prevention of cardiac hypertrophy, and/or neointima formation or restenosis. Further, the kit may comprise information, illustrations and/or instructions pertaining to the use of the derivative of angiotensin I.

The present invention also provides a pharmaceutical composition comprising an effective amount of at least one derivative of angiotensin I, with the exclusion of des-aspartate-angiotensin I, and a pharmaceutically acceptable carrier, excipient, diluent and/or carrier. The pharmaceutical composition may also comprise at least one pharmaceutical agent. A pharmaceutical agent may be, for example, at least one angiotensin converting enzyme inhibitor, at least one angiotensin receptor antagonist, at least one type of stem cell, and the like. In particular, the pharmaceutical composition according to the invention is for use in the treatment and/or prevention of cardiac hypertrophy and/or neointima formation in a subject in need of such treatment and/or prevention

As used herein, “cardiac hypertrophy” is the enlargement of the heart or any part of the heart, due to the condition of high blood pressure or any other cause. “Neointima formation” is the formation of undifferentiated or multi-types of new tissue in blood vessels due to injury or any other cause and includes restenosis. “Restenosis” is the re-narrowing, as in of a blood vessel, for example, the re-narrowing of a coronary artery after angioplasty. As used herein, restenosis can also be due to any other cause. The terms “cardiac hypertrophy”, “neointima formation” and “restenosis” are used in the broadest sense.

An “effective amount” refers to an amount effective, at dosages and for periods of time necessary to achieve the desired therapeutic result, such as to prevent, inhibit or delay the onset of cardiac hypertrophy, and/or neointima formation or restenosis or ameliorate the symptoms of cardiac hypertrophy, and/or neointima formation or restenosis. The effective amount may vary according to various factors such as the disease state, age, sex, and weight of the individual. The effective amount may range from 10 to 500 μg/kg/day for mammalian patients or subjects. More specifically, the effective amount may range from 50 to 250 μg/kg/day. Yet more specifically, the effective amount is about 150 μg/kg/day for cardiac hypertrophy and about 200 mg/kg/day for neointima formation in human patients.

A “derivative of angiotensin I” refers to any mutant, fragment, part or portion of angiotensin I, with the exclusions of des-aspartate-angiotensin I, but including molecules comprising single or multiple amino acid substitutions, deletions and/or insertions to angiotensin I and which inhibits, reduces or interferes with the activity or function of angiotensin I, or homologue, analogue or chemical equivalent thereof which is functionally equivalent in that it inhibits, reduces or otherwise interferes with the activity or functioning of angiotensin II.

Insertional amino acid sequence derivatives are those that include an addition of one or more amino acid residues. The addition may be introduced into a predetermined site or by random insertion with suitable screening of the resulting products. An amino acid insertional derivative of angiotensin I may include amino and/or carboxyl terminal fusions as well as intra-sequence insertions of single or multiple amino acids. Deletional derivatives are characterized by the removal of one or more amino acids from the sequence. Substitutional amino acid derivatives are those in which at least one residue in the sequence has been removed and a different residue inserted in its place.

A homologue of an angiotensin I derivative includes functionally, structurally or stereochemically similar polypeptides but with the exclusion of des-aspartate-angiotensin I, obtained from other species such as livestock animals and laboratory test animals, including rodents and primates.

An analogue of an angiotensin I derivative includes a mimotope, or peptide or analogue mimetic and includes molecules which contain non-naturally occurring amino acids as well as molecules which do not contain amino acids but nevertheless behaves as a functional equivalent, with the exclusion of des-aspartate-angiotensin I. Analogues contemplated herein include modifications to side chains, including deglycosylation or glycosylation, incorporation of unnatural amino acids and/or their derivatives during peptide synthesis and the use of crosslinkers and other methods which impose conformational constraints on the peptide molecule. Analogues also include angiotensin I derivatives coupled directly or indirectly to at least one modifying group while retaining the functionality of the derivative. Such modifications are well known in the art and include, for example, a derivative modified to alter a pharmacokinetic property, such as in vivo stability, bioavailability or half-life. The derivative may also be coupled to an additional therapeutic moiety or to a detectable substance.

Examples of unconventional (or unnatural) amino acids and/or their derivatives which may be incorporated during peptide synthesis include, but are not limited to, use of norleucine, 4-amino butyric acid, 4-amino-3-hydroxy-5-phenylpentanoic acid, 6-aminohexanoic acid, t-butylglycine, norvaline, phenylglycine, ornithine, sarcosine, 4-amino-3-hydroxy-6-methylheptanoic acid, 2-thienyl alanine and/or D-isomers of amino acids.

Crosslinkers may be used, for example, to stabilize three-dimensional conformations, using homo-bifunctional crosslinkers such as the bifunctional imido esters having (CH₂)ₙ spacer groups wherein n=1 to 6, glutaraldehyde, N-hydroxysuccinimide esters and hetero-bifunctional reagents which usually contain an amino-reactive moiety such as N-hydroxysuccinimide and another group specific-reactive moiety.

These types of modifications may be important to stabilize a derivative of angiotensin I, excluding des-aspartate-angiotensin I and -angiotensin II but including angiotensin IV. This may be important, for example, in the manufacture of a therapeutic composition or if angiotensin I derivative is used

Examples of non-conventional (or unnatural) amino acids contemplated by the present invention are presented in Table 1.

TABLE 1

| Non-conventional | |
|---|---|
| amino acid | Code |
| α-aminobutyric acid | Abu |
| α-amino-α-methylbutyrate | Mgabu |
| aminocyclopropane- | Cpro |
| carboxylate | |
| aminoisobutyric acid | Aib |
| aminonorbornyl- | Norb |
| carboxylate | |
| cyclohexylalanine | |
| cyclopentylalanine | Cpen |
| D-alanine | Dal |
| D-arginine | Darg |
| D-aspartic acid | Dasp |
| D-cysteine | Dcys |
| D-glutamine | Dgln |
| D-glutamic acid | Dglu |
| D-histidine | Dhis |
| D-isoleucine | Dile |
| D-leucine | Dleu |
| D-lysine | Dlys |
| D-methionine | Dmet |
| D-ornithine | Dorn |
| D-phenylalanine | Dphe |
| D-proline | Dpro |
| D-serine | Dser |
| D-threonine | Dthr |
| D-tryptophan | Dtrp |
| D-tyrosine | Dtyr |
| D-valine | Dval |
| D-α-methylalanine | Dmala |
| D-α-methylarginine | Dmarg |
| D-α-methylasparagine | Dmasn |
| D-α-methylaspartate | Dmasp |
| D-α-methylcysteine | Dmcys |
| D-α-methylglutamine | Dmgln |
| D-α-methylhistidine | Dmhis |
| D-α-methylisoleucine | Dmile |
| D-α-methylleucine | Dmleu |
| D-α-methyllysine | Dmlys |
| D-α-methylmethionine | Dmmet |
| D-α-methylornithine | morn |
| D-α-methylphenylalanine | Dmphe |
| D-α-methylproline | Dmpro |
| D-α-methylserine | Dmser |
| D-α-methylthreonine | Dmthr |
| D-α-methyltryptophan | Dmtrp |
| D-α-methyltyrosine | Dmty |
| D-α-methylvaline | Dmval |
| D-N-methylalanine | Dnmala |
| D-N-methylarginine | Dnmarg |
| D-N-methylasparagine | Dnmasn |
| D-N-methylaspartate | Dnmasp |
| D-N-methylcysteine | Dnmcys |
| D-N-methylglutamine | Dnmgln |
| D-N-methylglutamate | Dnmglu |
| D-N-methylhistidine | Dnmhis |
| D-N-methylisoleucine | Dnmile |
| D-N-methylleucine | Dnmleu |
| D-N-methyllysine | Dnmlys |
| N-methylcyclohexylalanine | Nmchexa |
| D-N-methylornithine | Dnmorn |
| N-methylglycine | Nala |
| N-methylaminoisobutyrate | Nmaib |
| N-(1-methylpropyl)glycine | Nile |
| N-(2-methylpropyl)glycine | Nleu |
| D-N-methyltryptophan | Dnmtrp |
| D-N-methyltyrosine | Dnmtyr |
| D-N-methylvaline | Dnmval |
| α-aminobutyric acid | Gabu |
| L-t-butylglycine | Tbug |
| L-ethylglycine | Etg |
| L-homophenylalanine | Hphe |
| L- -methylarginine | Marg |
| L- -methylaspartate | Masp |
| L- -methylcysteine | Mcys |
| L- -methylglutamine | Mgln |
| L- -methylhistidine | Mhis |
| L- -methylisoleucine | Mile |
| L- -methylleucine | Mleu |
| L- -methylmethionine | Mmet |
| L- -methylnorvaline | Mnva |
| L- -methylphenylalanine | Mphe |
| L- -methylserine | Mser |
| L- -methyltryptophan | Mtrp |
| L- -methylvaline | Mval |
| N-(N-(2,2-diphenylethyl) | Nnbhm |
| carbamylmethyl)glycine | |
| 1-carboxy-1-(2,2-diphenyl- | Nmbc |
| ethylamino)cyclopropane | |
| L-N-methylalanine | Nmala |
| L-N-methylarginine | Nmarg |
| L-N-methylasparagine | masn |
| L-N-methylaspartic acid | Nmasp |
| L-N-methylcysteine | Nmcys |
| L-N-methylglutamine | Nmgln |
| L-N-methylglutamic acid | Nmglu |
| Chexa L-N-methylhistidine | Nmhis |
| L-N-methylisolleucine | Nmile |
| L-N-methylleucine | Nmleu |
| L-N-methyllysine | Nmlys |
| L-N-methylmethionine | Nmmet |
| L-N-methylnorleucine | Nmnle |
| L-N-methylnorvaline | Nmnva |
| L-N-methylornithine | Nmorn |
| L-N-methylphenylalanine | Nmphe |
| L-N-methylproline | Nmpro |
| L-N-methylserine | Nmser |
| L-N-methylthreonine | Nmthr |
| L-N-methyltryptophan | Nmtrp |
| L-N-methyltyrosine | Nmtyr |
| L-N-methylvaline | Nmval |
| L-N-methylethylglycine | Nmetg |
| L-N-methyl-t-butylglycine | Nmtbug |
| L-norleucine | Nle |
| L-norvaline | Nva |
| α-methyl-aminoisobutyrate | Maib |
| α-methyl-α-aminobutyrate | Mgabu |
| α-methylcyclohexylalanine | Mchexa |
| α-methylcylcopentylalanine | Mcpen |
| α-methyl-α-napthylalanine | Manap |
| α-methylpenicillamine | Mpen |
| N-(4-aminobutyl)glycine | Nglu |
| N-(2-aminoethyl)glycine | Naeg |
| N-(3-aminopropyl)glycine | Norn |
| N-amino-α-methylbutyrate | Nmaabu |
| α-napthylalanine | Anap |
| N-benzylglycine | Nphe |
| N-(2-carbamylethyl)glycine | Ngln |
| N-(carbamylmethyl)glycine | Nasn |
| N-(2-carboxyethyl)glycine | Nglu |
| N-(carboxymethyl)glycine | Nasp |
| N-cyclobutylglycine | Ncbut |
| N-cycloheptylglycine | Nchep |
| N-cyclohexylglycine | Nchex |
| N-cyclodecylglycine | Ncdec |
| N-cylcododecylglycine | Ncdod |
| N-cyclooctylglycine | Ncoct |
| N-cyclopropylglycine | Ncpro |
| N-cycloundecylglycine | Ncund |
| N-(2,2-diphenylethyl)glycine | Nbhm |
| N-(3,3-diphenylpropyl)glycine | Nbhe |
| N-(3-guanidinopropyl)glycine | Narg |
| N-(1-hydroxyethyl)glycine | Nthr |
| N-(hydroxyethyl))glycine | Nser |
| N-(imidazolylethyl))glycine | Nhis |
| N-(3-indolylyethyl)glycine | Nhtrp |
| N-methyl-α-aminobutyrate | Nmgabu |
| D-N-methylmethionine | Dnmmet |
| N-methylcyclopentylalanine | Nmcpen |
| D-N-methylphenylalanine | Dnmphe |
| D-N-methylproline | Dnmpro |
| D-N-methylserine | Dnmser |
| D-N-methylthreonine | Dnmthr |
| N-(1-methylethyl)glycine | Nval |
| N-methyla-napthylalanine | Nmanap |
| N-methylpenicillamine | Nmpen |
| N-(p-hydroxyphenyl)glycine | Nhtyr |
| N-(thiomethyl)glycine | Ncys |
| penicillamine | Pen |
| L-α-methylalanine | Mala |
| L-α-methylasparagine | Masn |
| L-α-methyl-t-butylglycine | Mtbug |
| L-methylethylglycine | Metg |
| L-α-methylglutamate | Mglu |
| L-α-methylhomophenylalanine | Mhphe |
| N-(2-methylthioethyl)glycine | Nmet |
| L-α-methyllysine | Mlys |
| L-α-methylnorleucine | Mnle |
| L-α-methylornithine | Morn |
| L-α-methylproline | Mpro |
| L-α-methylthreonine | Mthr |
| L-α-methyltyrosine | Mtyr |
| L-N-methylhomophenylalanine | Nmhphe |
| N-(N-(3,3-diphenylpropyl) | Nnbhe |
| carbamylmethyl)glycine | |

A chemical equivalent of an angiotensin I derivative as described above, shares conformational or functional similarities and may not necessarily be derived from the derivative of angiotensin I. A chemical equivalent may be specifically designed to mimic certain physiochemical properties of a derivative of angiotensin I. Chemical equivalents may be chemically synthesized or may be detected following, for example, natural product screening of candidate compounds which can inhibit, reduce or otherwise interfere with the activity, or functioning of angiotensin II using assays described below.

A derivative of angiotensin I as defined herein may readily be made using synthetic techniques well known in the art, such as solid phase peptide synthesis and the like, or by recombinant DNA manipulations. Techniques for making substitution mutations at predetermined sites in DNA having known or partially known sequence are well known and include, for example, M13 mutagenesis. The manipulation of DNA sequence to produce variant proteins, which manifest as substitutional, insertional or deletional variants are conveniently described, for example, in Sambrook et al. (Cloning. A laboratory manual. Cold Spring Harbour Laboratory, Cold Spring Harbour, N.Y. (2001).

A derivative of angiotensin I according to the invention may be readily identified, for example, by its ability to act as an agonist on an indomethacin-sensitive angiotensin receptor or its ability to induce relaxation of a pre-contracted cardiac end of a rabbit pulmonary artery or its ability to attenuate angiotensin II-induced hypertrophy in cultured rat neonatal cardiomyocytes.

An example of such a derivative of angiotensin I in accordance with the present invention is angiotensin IV, or derivative, homologue, analogue or chemical equivalent thereof. The term derivative in this context has the same meaning as used in the context of angiotensin I as described above. Similarly, the terms homologue or analogue and chemical equivalent as used in this context has the same meaning as described above for angiotensin I derivative generally.

It is well known in the art that modifications and changes can be made to the structure of a peptide without substantially altering the biological function of that peptide. To this end, where angiotensin IV is derivatized by amino acid substitution, the amino acids are generally replaced by other amino acids having like properties, such as hydrophobicity, hydrophilicity, electronegativity, size, and the like. Amino acid substitutions are typically of single residues. Amino acid insertions will usually be in the order of about 1 to 6 amino acid residues and deletions will range from about 1 to 6 residues.

Reference herein to angiotensin I derivative and angiotensin IV should be read as including reference to all functionally equivalent forms, including, by way of example, isoforms, monomeric, dimeric and multimeric forms.

In accordance with the present invention, an effective amount of the derivative of angiotensin I such as but not limited to angiotensin IV or a derivative, homologue, analogue or chemical equivalent thereof or a medicament or pharmaceutical composition containing the same, as described below, is administered in a solid or liquid form, to a subject, such as a human patient, via any acceptable method known in the art, either singly or in combination with other pharmaceutical agents. “Pharmaceutical agent” means any diagnostic and/or therapeutic drug or combination of drugs that has the property of assisting the medical or pharmaceutical use of the derivative of angiotensin I according to the invention. In particular, “pharmaceutical agent” means any diagnostic and/or therapeutic drug or combination of drugs that has the property of assisting in the treatment and/or prevention of cardiac hypertrophy and/or neointima formation. Such pharmaceutical agents include angiotensin converting enzyme inhibitors such as captopril or other angiotensin receptor antagonists such as losartan, or stem cells of any types or origin.

The compound, composition and/or medicament according to the invention may be administered orally, by suppository, or parenterally (e.g. intramuscularly, intravenously, subcutaneously or intradermally), and in the form of either solid or liquid dosage including tablets, suspensions, or solutions, as is discussed in more detail below. The administration may be conducted in single dosage form with continuous therapy or in single dose therapy ad libitum.

Useful pharmaceutical carrier, excipient, diluent and/or adjuvant for the preparation of the pharmaceutical composition or medicament of the invention are well know to a skilled person and may be solids, liquids or mixtures thereof; thus, the compositions may take the form of tablets, pills, capsules, powders, enterically coated or other protected formulations, sustained release formulations, erodible formulations, implantable devices or components thereof, microsphere formulations, solutions, suspensions, elixirs, aerosols and the like.

Water, saline, aqueous dextrose, and glycols are preferred liquid carriers, particularly (when isotonic) for injectable solutions. The carrier may be selected from various oils including those of petroleum, animal, vegetable or synthetic origin, for example, peanut oil, soybean oil, mineral oil, sesame oil, and the like. Suitable pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk, glycerol, propylene glycol, water, ethanol, and the like. Other pharmaceutically acceptable carrier, excipient, diluent and/or adjuvant will be apparent to one skilled in the art. The composition may be subjected to conventional pharmaceutical expedients such as sterilization and may contain conventional pharmaceutical additives such as preservatives, stabilizing agents, wetting or emulsifying agents, salts for adjusting osmotic pressure, buffers and the like. Suitable pharmaceutical carriers and their formulations are described in Martin, “Remington's Pharmaceutical Sciences”, 15^{th} Ed.; Mack Publishing Co., Easton (1975); see, e.g. pp. 1405-1412 and pp 1461-1487. Such compositions will, in general, contain an effective amount of the active compound together with a suitable amount of at least one pharmaceutically acceptable carrier, excipient, diluent and/or adjuvant so as to prepare the proper dosage form for proper administration to the host.

In the practice of the therapeutic methods of the invention, the particular dosage of pharmaceutical composition to be administered to the subject will depend on a variety of consideration including the stage of the disease or condition, the severity thereof, the schedule of administration, the age and physical characteristics of the subject, and so forth. Proper dosages may be established using clinical approaches familiar to the medicinal arts.

Although the present invention is particularly exemplified herein in relation to rats, it is understood that the present invention extends to the use of angiotensin I derivatives according to the invention in any mammal subject including, but not limited to, humans, mice, rabbits, livestock animals and primates.

Having now generally described the invention, the same will be more readily understood through reference to the following examples which are provided by way of illustration, and are not intended to be limiting of the present invention.

### EXAMPLES

### Example 1

### Source of Materials

Angiotensin IV, as an example of an Angiotensin I derivative, was obtained from Bachem (Dubendorf, Switzerland). Angiotensin IV can be prepared by techniques well known in the art. Adult Sprague Dawley (SD) rats (200-220 g for cardiac hypertrophy experiment, and 340-360 g for the neointima formation experiment) were obtained from the Animal Center, National University of Singapore.

### Example 2

### Induction of Cardiac Hypertrophy

The experimental protocol for induction of cardiac hypertrophy in rats was carried out as described by Everett et al (Hypertension, 23:587-592 (1994)). In this procedure, each rat was anaesthetized with 7% w/v chloral hydrate (0.35 g/kg, intraperitoneally). An incision was made in the ventral abdominal wall to access the suprarenal portion of the abdominal aorta. This portion of the abdominal aorta was dissected free and a blunt 23-guage needle was placed adjacent to the aorta. A ligature was placed around the blunt needle and the aorta. The blunt needle was then removed, leaving the aorta constricted to the size of the needle. The resulting coarctation resisted the normal flow of blood from the heart to the lower portion of the body and placed an extra load on the heart. This extra load causes hypertrophy of the heart, especially the left ventricle.

### Example 3

### Treatment with Angiotensin IV and Measurement of Cardiac Hypertrophy

Following surgery, each animal was placed in a cage. The animals had access to water and rat chow ad libitum. The animals were randomly divided into the control group and treatment group. Each group consisted of 10 animals. The treatment group was orally administered various doses of angiotensin IV (95-380 nmoles/kg/day or 74-294 μg/kg/day) dissolved in 0.5 ml saline for four days commencing on the day of surgery. Control animals with coarcted abdominal aorta were administered saline instead of the angiotensin IV solution. Sham animals were animals that underwent the same surgical operations but their aortas were not coarcted.

On the fifth day following surgery, animals were anaesthetised as before. The heart of each animal was then excised, the ventricles dissected and the weight of the ventricles was determined. The index of the ventricle weight (in mg) over the body weight of the animal (in g) was taken as the index of hypertrophy. For sham-operated animals the index was around 2.6, for aorta-coarcted animas the index was above 3.7.

### Example 4

### Effect of Angiotensin IV on Cardiac Hypertrophy

The results of the study are summarized in Table 2. Data were expressed as mean ±SEM. Significant differences were determined by one-way ANOVA and post hoc Newman Kleuf test. The accepted level of significance was p<0.05. Angiotensin IV, as an example of a derivative of angiotensin I, was shown to be an effective agent in attenuating the index of hypertrophy in experimentally-induced cardiac hypertrophic rats. The effect was dose-dependent and significant anti-cardiac hypertrophic action was brought about by an oral dose of 190 nmoles/kg/day (or 147 μg/kg/day).

TABLE 2
Effects of angiotensin IV on cardiac hypertrophy in rats

| Dose (nmole/kg/day for 4 | Hypertrophy Index |
|---|---|
| days) | (ventricle weight in mg/body weight in g) |
| Sham | 2.64 ± 0.04 |
| Control | 3.83 ± 0.06 |
| 95 | 3.73 ± 0.04 |
| 190 | *3.23 ± 0.07 |
| 380 | *3.40 ± 0.07 |

Each value is a mean ± SEM obtained from 10 individual rats.
*Significantly different from the Control (p < 0.05).

### Example 5

### Induction of Neointima Growth

SD rats were subjected to left carotid artery injury by the balloon technique according to the method described by Indolfi et al (Circulation, 92:1230-1235 (1995)). In this procedure, rats were anesthetized with chloral hydrate (0.35 g/kg) and a balloon catheter (2F Fogarty, Edwards Laboratories) was introduced through the left external carotid artery into the common carotid artery. The balloon was inflated to a pressure of 2.2 kg/cm² by compressed carbogen gas mixture (95% O₂ and 5% CO₂) and passed three times (three cycles) along the common carotid artery. The catheter was removed, the left external carotid artery was ligated, and the wound was closed. Formation of neointima in the catheter-injured carotid artery occurred and slowed considerably after 14 days (Clowes and Clowes, Lab. Invest., 52:611-616 (1985)). The right common carotid artery was left intact and served as the control artery.

### Example 6

### Treatment with Angiotensin IV and Quantitation of Neointima Formation

Following the surgery, each animal was placed in a cage. The animals had access to water and rat chow ad libitum. The animals were randomly divided into the control group and treatment group. Each group consisted of 6 animals. The treatment group was orally administered various doses of angiotensin IV (60-360 nmoles/kg/day or 46.5-279 μg/kg/day) dissolved in 0.5 ml saline for 13 days commencing on the day of surgery. Control animals were balloon catheterized animals that were administered saline instead of the angiotensin IV solution.

On the fourteenth day following balloon catheterization, animals were anesthetized as before and both the left and right common arteries of each rat were fixed by perfusion at 120 mm Hg with 100 ml of saline followed by 250 ml of 0.1 M phosphate buffer (pH 7.4) containing 4% paraformaldehyde and 1% glutaralaldehyde and processed for paraffin embedment. Sections of 10 μm thickness were prepared and stained with toluidine blue. Twenty of such sections were cut from the midportion of the artery towards the distal end and used for morphometric evaluation of neointima formation. The area of the medial smooth muscle cells, lumen, and neointima of each section was morphometrically quantitated using an image analysis system consisting of a BX40 light microscope (Olympus, Japan) fitted with a KY-F55B color video camera (JVC, Japan) and a Pentium 166 MHz/MMX microcomputer (Datamini, Singapore) installed with an Image Pro Plus 3.0 System (Media Cybernetics, USA) for Windows 95™. The extent of neointima formation was expressed as a percentage of occlusion of the lumen by the neointima.

### Example 7

### Effect of Angiotensin IV on Neointima formation

The results of the study are summarized in Table 3. Angiotensin IV, as an example of a derivative of angiotensin I, has been found to be an effective agent in preventing the formation of neointima resulting from balloon catheterization. The anti-neointima action is dose-dependent and its maximum action is brought about by an oral dose of 240 nmoles/kg/day (or 186 μg/kg/day) for 13 days. However, angiotensin IV has no significant effect on the thickness of the medial muscle layer.

TABLE 3
Effects of angiotensin IV on neointima formation

| Dose (nmole/kg/day for 13 | % of Lumen Occlusion by Neointima |
|---|---|
| days) | (in catheter-injured carotid artery) |
| Control | 55 ± 2 |
| 60 | 52 ± 5 |
| 120 | 48 ± 1 |
| 240 | *43 ± 2 |
| 360 | *44 ± 2 |

Each value is a mean ± SEM obtained from 6 individual rats.
*Significantly different from the Control (p < 0.05).

All references cited herein are fully incorporated by reference. Having now described the invention, it will be understood by those skilled in the art that various modifications can be made to the described embodiments without departing from the scope of the invention. Such modifications are intended to be within the scope of the invention.

### REFERENCES

Baker and Aceto (Am. J. Physiol., 259:H610-H618 (1990)
Clowes and Clowes, Lab. Invest., 52:611-616 (1985)
Daemen et al, Circ. Res., 68:450-456 (1991)
Dostal and Baker, Am. J. Hypertens., 5:276-280 (1991)
Everett et al (Hypertension, 23:587-592 (1994)
Indolfi et al (Circulation, 92:1230-1235 (1995)
Moeller et al (Regul. Pept., 83:25-30 (1999)
Osterrieder et al, Hypertension, 18:II-60-II-64 (1991)
Swanson et al, Regul. Pept., 40:409-419 (1992)
von Bohlen and Halbach, Cell Tissue Res., 311:1-9 (2003)
Wang et al (Clin. Sci., 88:557-562 (1995)

1-56. (canceled)

57. A method for the treatment and/or prevention of cardiac hypertrophy and/or neointima formation in a subject in need of such treatment and/or prevention comprising administering to the subject an effective amount of at least one molecule selected from angiotensin IV, a derivative, homologue, analogue and/or chemical equivalent of angiotensin IV.

58. The method according to claim 57, wherein neointima formation comprises restenosis.

59. The method according to claim 57, wherein the subject is a human patient.

60. The method according to claim 57, wherein the effective amount is 10 to 500 μg/kg/day.

61. The method according to claim 57, wherein the effective amount is 50 to 250 μg/kg/day.

62. The method according to claim 57, wherein the effective amount is about 150 μg/kg/day for the treatment and/or prevention of cardiac hypertrophy.

63. The method according to claim 57, wherein the effective amount is about 200 μg/kg/day for the treatment and/or prevention of neointima formation and/or restenosis.

64. The method according to any one of claims 57 through 63, wherein the derivative is administered in solid or liquid form.

65. The method according to claim 57, wherein the derivative is administered in conjunction with at least one pharmaceutical agent.

66. The method according to claim 65, wherein the at least one pharmaceutical agent is an angiotensin converting enzyme inhibitor.

67. The method according to claim 65, wherein the at least one pharmaceutical agent is an angiotensin receptor antagonist.

68. The method according to claim 65, wherein the at least one pharmaceutical agent is a type of stem cell.

69. A kit comprising at least one molecule selected from angiotensin IV, derivative, homologue, analogue and/or chemical equivalent of angiotensin IV, wherein the kit is for the treatment and/or prevention of cardiac hypertrophy and/or neointima formation.

70. The kit according to claim 69, wherein neointima formation includes restenosis.

71. A method for the treatment and/or prevention of cardiac hypertrophy and/or neointima formation in a subject in need of such treatment and/or prevention comprising administering to the subject an effective amount of at least one derivative of angiotensin I, with the exclusion of des-aspartate-angiotensin I, angiotensin-(1-7), angiotensin-(1-9) and angiotensin-(3-7).

72. The method according to claim 71, wherein the derivative is selected from angiotensin IV, a derivative, homologue, analogue and/or chemical equivalent of angiotensin IV.

73. The method according to claim 71, wherein neointima formation comprises restenosis.
